(19) United States
(12) Patent Application Publication
(10) Pub. No.: US 2025/0052671 A1
(43) Pub. Date: Feb. 13, 2025

(54) Method of Measuring a Methane Destruction and Removal Efficiency of an Elevated Flare

(71) Applicant: Cimarron Energy, Inc., Houston, TX (US)

(72) Inventor: Jianhui Hong, Houston, TX (US)

(21) Appl. No.: 18/800,496

(22) Filed: Aug. 12, 2024

Related U.S. Application Data
(60) Provisional application No. 63/532,101, filed on Aug. 11, 2023.
(60) Provisional application No. 63/532,477, filed on Aug. 14, 2023.

(51) Int. Cl. G01N 21/3504; G01N 21/35; G01N 33/00

(52) U.S. Cl. CPC G01N 21/3504; G01N 33/0036; G01N 2021/3531; G01N 2021/3595

(57) ABSTRACT

A method and related system, including a processing unit, of determining a methane destruction and removal efficiency (DRE) of an elevated flare receiving at least one waste gas stream and having a flame and a plume. The method includes the steps of: determining a signal strength for each carbon-containing species in the plume using PFTIR sensing over a period of time; measuring a volumetric flow rate for each of the at least one waste gas stream during the period of time; calculating a methane carbon fraction for the at least one waste gas stream using a predetermined gas composition for each of the at least one waste gas stream; calculating a methane carbon fraction for the plume; and calculating the methane DRE using the calculated methane carbon fraction for each of the at least one waste gas stream and the calculated methane carbon fraction for the plume.

This invention was made with government support under contract number DE-AR0001530 awarded by the United States Department of Energy. The government has certain rights in the invention.

This application claims the benefit of U.S. Provisional Patent Application Nos. 63/532,101, filed Aug. 11, 2023, and 63/532,477, filed Aug. 14, 2023, the disclosures of which are incorporated herein by reference.

### TECHNICAL FIELD

The invention generally relates to elevated flares and, more specifically, to a method of measuring a methane destruction and removal efficiency (DRE) of the elevated flares.

### BACKGROUND

Gas flares are commonly located at production facilities, refineries, processing plants and the like for disposing of flammable associated waste gases and other flammable gas streams that are diverted due to venting requirements, shut-downs, upsets and/or emergencies. As safety devices and pollution control devices, it is desired and generally required that such flares operate in a manner that eliminates or significantly reduces smoke in order to minimize, if not eliminate, the release of black soot into the atmosphere. More specifically, flares are designed and operated to meet regulatory standards which generally require them to have a flare destruction and removal efficiency (DRE) of at least 98% with the most recent regulations issued in 2024 further affecting the 98% assumed DRE figure. This has largely been achieved by admixing the flammable gas to be discharged and burned, otherwise known as flare gas, with enough air to sufficiently oxidize the gas. It should be noted that flare DRE is defined herein as the fraction of combustible compounds destroyed or removed, in other words, the fraction of combustible compounds converted to products different from the original molecules of the combustible compounds.

In the past, measuring the combustion products in a flare plume from an industrial-sized flare was difficult. This is due primarily to the fact that a typical flare apparatus includes a flare stack elevating a flare tip mounted on the flare stack high above the ground. In such arrangements, the flare flame is not confined within a solid boundary. Ambient cross winds often act on the flare flame creating unsteady and turbulent flame patterns which increase the difficulty of measuring DRE, even for methods using sampling probes. Recent technological advances, however, have produced remote sensing capabilities including the ability to measure combustion products such as carbon dioxide, carbon monoxide, and select hydrocarbons without the challenges introduced by physically sampling a flare plume. One such instrument utilizes Passive Fourier transform infrared (PFTIR) and is commonly used to measure flare DRE.

The PFTIR instrument works by characterizing a flare plume's chemical make-up.

More specifically, the PFTIR instrument measures an amount of energy radiated from the plume from which all species in the plume can be identified and quantified, in units of parts-per-million (ppm)×meter (m) (concentration level×path length). The species may include carbon-containing species, such as, carbon monoxide, carbon dioxide, methane, and other hydrocarbons, and non-carbon-containing species including, for example, nitrogen oxides and ammonia. With this information, a flare's combustion efficiency (CE) can be measured or determined. One method of measuring CE of a flare using a PFTIR instrument is described in U.S. Pat. No. 9,146,195 to Spellicy which is incorporated herein in its entirety by reference.

According to the aforementioned patent, flare CE can be calculated using the following formula:

$$Flare CE = \frac{[ {CO}_{2} ]}{[ CO ] + [ {CO}_{2} ] + [ T HC ] + [ soot ]} . \qquad ( Equation 1 )$$

The advantage of the method described in the incorporated patent is its ability to measure the CE of a flare without prior knowledge of the composition of the flare gas. Rather, the PFTIR instrument uses signals measured from a hot flue gas, measurements of a background radiance, and measurements of various calibrators, namely, a hot infrared source and a cold infrared source to determine the flare CE. According to the aforementioned patent, the PFTIR instrument can also calculate a flare DRE using the following formula:

$$Flare DRE = \frac{[ {CO}_{2} ] + [ CO ]}{[ CO ] + [ {CO}_{2} ] + [ T HC ] + [ soot ]} . \qquad ( Equation 2 )$$

While the method of using PFTIR instruments described in the incorporated patent provides one means of measuring flare DRE, there is no description or teaching of measuring methane DRE for a flare or any other specific gas of interest. In other words, the disclosed PFTIR instrument, or method, does not measure the destruction and removal efficiency of methane for the flare. Methane DRE is defined herein as the fraction of methane that is converted to anything other than methane.

Even more, the disclosed means of measuring flare DRE in the incorporated patent assumes that hydrocarbons in the flare gases are only converted to either CO or CO₂. As a result, if methane or any other hydrocarbons from the flare gas were broken down and turned into other hydrocarbons, such as ethylene or formaldehyde, in the flame before being vented to the atmosphere, the methane component would not be counted as destroyed or removed for purposes of the DRE calculation.

The importance of measuring or determining methane DRE for a flare gained significant importance with the enactment of the Inflation Reduction Act of 2022 (IRA). Among other provisions, IRA includes a charge or fee on methane emissions from selected entities in the oil and gas industry that are required to report their greenhouse gas (GHG) emissions, such as methane, to the Environmental Protection Agency's (EPA's) Greenhouse Gas Emissions Reporting Program (GHGRP).

Methane (CH₄) is a primary component of natural gas, which can be used as either a fuel or as a feedstock for the chemical industry. Natural gas is generally produced from geologic formations in the ground through drilling and extraction activities. As natural gas travels through the interconnected systems of exploration, production, processing, storage, and/or transmission, that deliver natural gas from a wellhead to a consumer, methane emissions may be released into the atmosphere in a variety of ways, including via flaring. Flaring of excess natural gas at a petroleum production site, for example, can result in a release of uncombusted methane. Given the parameters of the IRA with regard to methane and other GHG emissions, a need exists for a means of measuring methane DRE for a flare.

In the following description, there are shown and described several systems and related methods for measuring methane DRE for elevated flares. As it should be realized, the disclosed systems and methods are capable of other, different embodiments and their several details are capable of modification in various, obvious aspects all without departing from the methods as set forth and described in the following claims. Accordingly, the drawings and descriptions should be regarded as illustrative in nature and not as restrictive.

### SUMMARY OF THE INVENTION

In accordance with the purposes and benefits described herein, a method of determining a methane destruction and removal efficiency (DRE) of an elevated flare receiving at least one waste gas stream and having a flame and a plume includes the steps of: determining, by a processing unit, a signal strength for each carbon-containing species in the plume using passive Fourier transform infrared remote sensing over a period of time; measuring a volumetric flow rate for each of the at least one waste gas stream during the period of time; calculating, by the processing unit, a methane carbon fraction for the at least one waste gas stream using a predetermined gas composition for each of the at least one waste gas stream; calculating, by the processing unit, a methane carbon fraction for the plume; and calculating, by the processing unit, the methane DRE using the calculated methane carbon fraction for the at least one waste gas stream and the calculated methane carbon fraction for the plume.

In another possible embodiment, the at least one waste gas stream is conducted to the flame at least partially through the flare.

In yet another possible embodiment, the step of determining a gas composition for each of the at least one waste gas stream.

In still another possible embodiment, the step of calculating a methane carbon fraction for the at least one waste gas stream, by the processing unit, utilizes Equation 8 if the at least one waste gas stream includes a single waste gas stream, Equation 9 if the at least one waste gas stream includes two waste gas streams, and Equation 10 if the at least one waste gas stream includes more than two waste gas streams.

In yet one other possible embodiment, the step of calculating a methane carbon fraction for the plume, by the processing unit, utilizes Equation 5.

In another possible embodiment, the step of determining a signal strength for each carbon-containing species in the plume includes the steps of measuring radiation data based on radiation from the plume and a background radiance, calculating, by the processing unit, a difference of a measured radiance of the plume and a measured background radiance, and calculating, by the processing unit, based on the difference, a transmissivity of the plume such that the calculated transmissivity of the plume is defined for flare combustion temperatures.

In one other possible embodiment, the step of determining a signal strength for each carbon-containing species in the plume includes the steps of calculating a correction factor based on atmospheric transmissivity, and wherein the step of calculating transmissivity of the plume further includes the step of calculating, based on the calculated correction factor, the transmissivity of the plume.

In another aspect of the invention, a method of determining a methane destruction and removal efficiency (DRE) of an elevated flare receiving at least two waste gas streams and having a flame and a plume, utilizing a computing device, includes the steps of: determining a gas composition for each of the at least one waste gas stream; determining a signal strength for each carbon-containing species in the plume using passive Fourier transform infrared remote sensing; measuring a volumetric flow rate for each of the at least two waste gas streams; calculating a methane carbon fraction for the at least one waste gas stream using the determined gas composition for each of the at least two waste gas streams; calculating a methane carbon fraction for the plume; and calculating the methane DRE using the calculated methane carbon fraction for each of the at least two waste gas streams and the calculated methane carbon fraction for the plume.

In another possible embodiment, each of the at least two waste gas streams is conducted to the flame at least partially through the flare.

In yet another possible embodiment, the method further includes the step of mixing the at least two waste gas streams with air conducted through the flare.

In still another possible embodiment, the step of determining a gas composition for each of the at least two waste gas streams includes the steps of collecting waste gas samples for each of the at least two waste gas streams and analyzing the collected waste gas samples before performing the step of determining a signal strength for each carbon-containing species in the plume using passive Fourier transform infrared remote sensing.

In yet still one other possible methods, the step of calculating a methane carbon fraction for the at least two waste gas streams utilizes Equation 9 if the at least two waste gas streams include two waste gas streams and Equation 10 if the at least two waste gas streams include more than two waste gas streams.

In another possible embodiment, the step of calculating a methane carbon fraction for the plume utilizes Equation 5.

In still one additional possible embodiment, the step of determining a signal strength for each carbon-containing species in the plume includes the steps of measuring radiation data based on radiation from the plume and a background radiance, and calculating a transmissivity of the plume based on a calculated difference of a measured radiance of the plume and a measured background radiance.

In accordance with another aspect of the invention, a system for determining a methane destruction and removal efficiency (DRE) of an elevated flare is provided. The system includes a computing device and a computer-readable medium coupled to the computing device having instructions stored thereon which, when executed by the computing device, cause the computing device to perform certain operations, including: calculating a methane carbon fraction for the plume using a determined signal strength for each carbon-containing species in the plume using passive Fourier transform infrared remote sensing over a period of time; calculating a methane carbon fraction for at least one waste gas stream conducted at least partially through an elevated flare having a flame and a plume using a predetermined gas composition for each of the at least one waste gas stream and a measured volumetric flow rate for each of the at least one waste gas stream over the period of time; and calculating the methane DRE using the calculated methane carbon fraction for each of the at least one waste gas stream and the calculated methane carbon fraction for the plume.

In another possible embodiment, the system further includes a flow meter for each of the at least one waste gas stream for measuring a volumetric flow rate for each of the at least one waste gas stream.

In still another possible embodiment, the system further includes an PFTIR instrument for determining a signal strength for each carbon-containing species in the plume using passive Fourier transform infrared remote sensing over a period of time.

In yet another possible embodiment, the operation of calculating a methane carbon fraction for the at least one waste gas stream utilizes Equation 8 if the at least one waste gas stream includes a single waste gas stream, Equation 9 if the at least one waste gas stream includes two waste gas streams, and Equation 10 if the at least one waste gas stream includes more than two waste gas streams.

In still one other possible embodiment, the operation of calculating a methane carbon fraction for the plume, by the processing unit, utilizes Equation 5.

In the following description, there are shown and described several embodiments of a system and related methods for determining a methane destruction and removal efficiency of an elevated flare receiving at least one waste gas stream and having a flame and a plume. As it should be realized, the systems and related methods of determining a methane destruction and removal efficiency of an elevated flare are capable of other, different embodiments and their several details are capable of modification in various, obvious aspects all without departing from the hybrid flare apparatus as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the inventive devices and related methods of determining a methane DRE of an elevated flare and together with the description serve to explain certain principles thereof. In the drawing figures:

FIG. 1 is an elevated tandem flare for combustion of both a high-pressure waste gas stream and a low-pressure waste gas stream; and

FIG. 2 is an illustration of how a PFTIR instrument is used to measure methane DRE from an elevated tandem flare plume and a computing device used for the same purpose.

Reference will now be made in detail to the present embodiments of the inventive system and related methods of determining a methane DRE of an elevated flare, examples of which are illustrated in the accompanying drawing figures, wherein like numerals are used to represent like elements.

### DETAILED DESCRIPTION

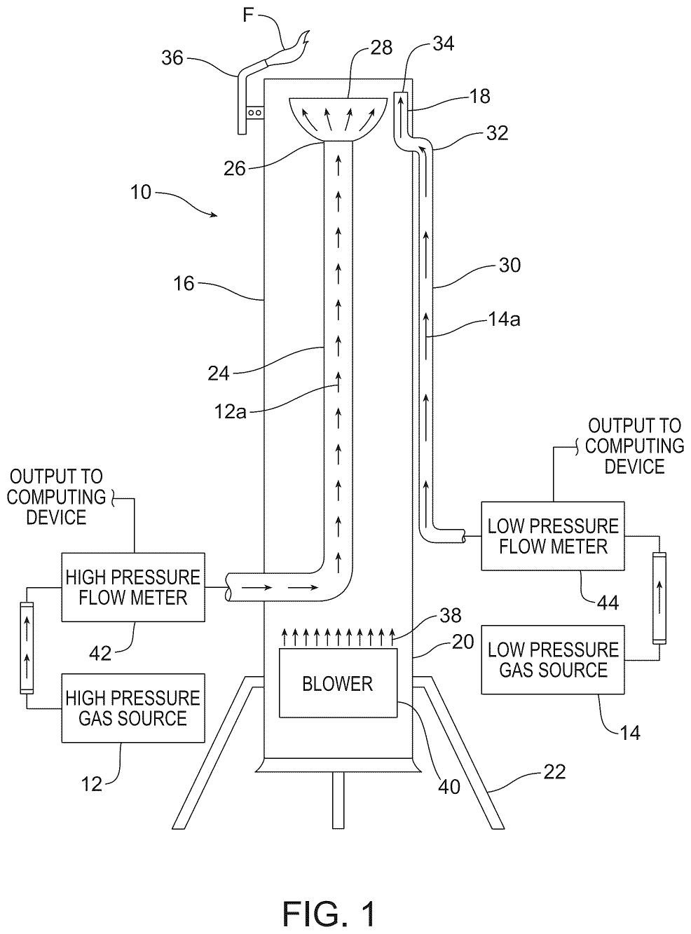

Reference is made to FIG. 1 which illustrates an elevated tandem flare 10, generally known in the art for combustion of large flows of waste gas, having a high-pressure waste gas stream 12a from a high-pressure source 12 and a tandem, low-pressure waste gas stream 14a from a low-pressure source 14. The elevated flare 10 includes a vertically oriented flare body or stack 16 having a proximal portion 18 defining an orifice and a distal portion 20. The proximal portion 20 is closest to the ground relative to the elevated distal portion 18. In the described embodiment, at least three legs 22, support the flare stack 16 in a generally vertical manner as is known in the art. In other embodiments, the flare 10 may be oriented at an angle if desired. For example, a flare used on an offshore platform may be oriented 45 degrees from vertical in order to project the flare flame away from the platform. It will also be appreciated by those familiar with the environments in which flares operate that wind and other factors may cause the flare to be angled slightly during operation.

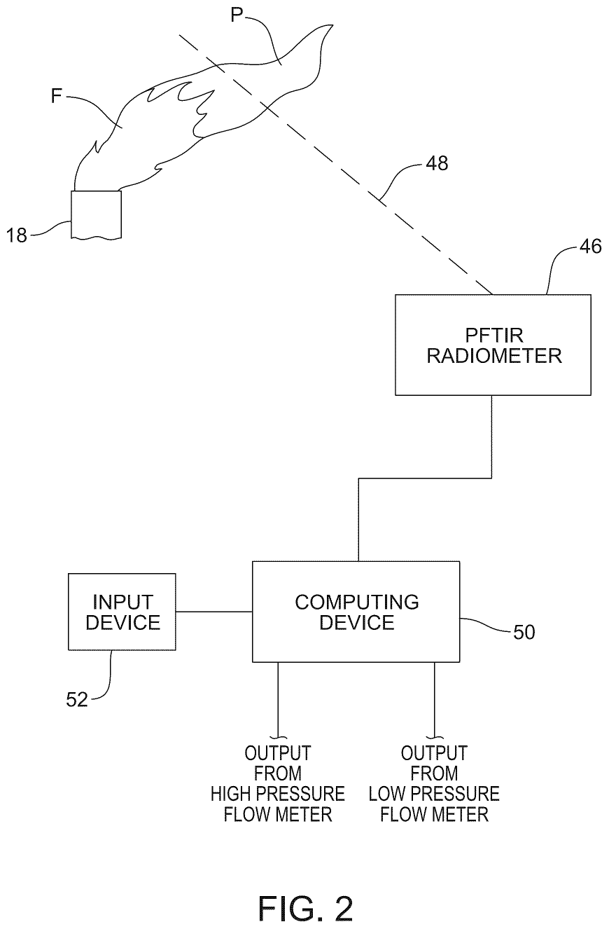

A high-pressure gas (HP gas) riser 24 extends within the flare stack 16 and is connected at an upper end 26 to a fixed-orifice nozzle 28. As illustrated by arrows 12a in FIG. 1, the high-pressure waste gas stream 12a flows upward through the high-pressure riser 24 and out of the nozzle 28. An external, low-pressure gas (LP gas) riser 30 extends vertically and adjacent the flare stack 16 as shown. In the described embodiment, the low-pressure waste gas stream 14a flows upward within the LP gas riser 30. An upper portion 32 of the riser 30 curves or bends and extends through the distal portion 18 of the flare stack 16. In the described embodiment, the LP gas riser 30 bends a second time and extends further vertically, but within the flare stack 16. The LP gas riser 30 terminates at a low-pressure orifice 34 adjacent to the nozzle 28. An ignitor 36 is supported by the flare stack 16 and creates a pilot flame (F) which ignites one or both of the high-pressure and/or low-pressure waste gas streams 12a, 14a exiting the respective risers 24 and 30 as is known in the art. The result is a flare flame F and a hot plume P as shown in FIG. 2.

To facilitate combustion of the high-pressure and low-pressure waste gas streams 12a, 14a and ensure smokeless combustion, auxiliary air (generally illustrated by arrows 38) is fed into the flare stack 16 by a ground-based fan or blower 40 illustrated in schematic form. The auxiliary air combines with the waste gas stream(s) 12a and 14a exiting the HP gas riser 24 and LP gas riser 30 causing turbulence therein. This improves mixing of the waste gas stream(s) 12a, 14a with air and ultimately combustion efficiency. Such air-assisted flares are generally known in the art. In other embodiments, gas-assisted flares may similarly be utilized to improve combustion efficiency of the HP and/or LP waste gas streams. Although the described flare embodiment includes two waste gas streams, other air-assisted flare embodiments could accommodate three waste gas streams, including, for example, an HP, an LP, and a third waste gas stream.

The high-pressure waste gas stream 12a is typically an associated gas. It should be noted that the term HP gas is an industry term and does not necessarily mean that the waste gas is exerting a high pressure on the riser 24 at all times. The gas is often rich in methane such that methane is typically a major component of the gas. As shown in FIG. 1, the HP waste gas stream 12a is conducted to the flare 10 through a high pressure flow meter 42 for disposal of the high-pressure gas. One exemplary composition of a typical HP waste gas stream is shown in Table 1 below wherein methane makes up around two-thirds of the gas as shown in column 5. Columns 6 and 7 are described in detail below.

TABLE 1
Typical composition of a high-pressure waste gas (flare waste
gas Stream No. 1, corresponding to volumetric flow rate Q₁).

| | | | | Volume | | |
|---|---|---|---|---|---|---|
| | | | | Fraction | x × | Fc(CH₄, |
| Name | CₓH_{y} | x | y | F₁(CₓH_{y}) | F₁(CₓH_{y}) | flare gas 1) |
| Methane | CH₄ | 1 | 4 | 0.67 | 0.67 | 0.431 |
| Ethane | C₂H₆ | 2 | 6 | 0.149 | 0.298 | |
| Propane | C₃H₈ | 3 | 8 | 0.1 | 0.3 | |
| Butane | C₄H₁₀ | 4 | 10 | 0.042 | 0.168 | |
| Pentane | C₅H₁₂ | 5 | 12 | 0.013 | 0.065 | |
| Hexane | C₆H₁₄ | 6 | 14 | 0.004 | 0.024 | |
| Heptane | C₇H₁₆ | 7 | 16 | 0.003 | 0.021 | |
| Carbon | CO₂ | 1 | 0 | 0.007 | 0.007 | |
| Dioxide | | | | | | |
| Nitrogen | N₂ | 0 | 0 | 0.012 | 0 | |
| Total | | | | 1 | 1.553 | |

The LP waste gas stream 14a is typically a tank vapor, i.e., a vapor above a liquid crude oil within an LP waste gas source 14, such as a storage tank. Such storage tanks are typically constructed using thin gauge sheet metal. As such, they are not designed to withstand high internal pressure or high vacuum. When the tank vapor pressure meets or exceeds a certain value (for example, 0.5 psig), a pressure relief valve is opened to allow a release of the tank vapor. The released vapor forms at least a part of the LP waste gas stream 14a and is similarly conducted to the flare 10 through a low pressure flow meter 44 for disposal of the low-pressure gas as shown in FIG. 1. One exemplary composition of a typical LP waste gas stream is shown in Table 2 wherein methane makes up a lesser portion at just under two-tenths of the gas as shown in column 5. Columns 6 and 7 are described in detail below.

TABLE 2
Typical composition of a low pressure waste gas (flare waste
gas Stream No. 2, corresponding to volumetric flow rate Q₂).

| | | | | Volume | | |
|---|---|---|---|---|---|---|
| | | | | Fraction | x × | Fc(CH₄, |
| Name | CₓH_{y} | x | y | F₂(CₓH_{y}) | F₂(CₓH_{y}) | flare gas 2) |
| Methane | CH₄ | 1 | 4 | 0.17 | 0.17 | 0.060 |
| Ethane | C₂H₆ | 2 | 6 | 0.23 | 0.46 | |
| Propane | C₃H₈ | 3 | 8 | 0.31 | 0.93 | |
| Butane | C₄H₁₀ | 4 | 10 | 0.18 | 0.72 | |
| Pentane | C₅H₁₂ | 5 | 12 | 0.065 | 0.325 | |
| Hexane | C₆H₁₄ | 6 | 14 | 0.017 | 0.102 | |
| Heptane | C₇H₁₆ | 7 | 16 | 0.015 | 0.105 | |
| Carbon | CO₂ | 1 | 0 | 0.006 | 0.006 | |
| Dioxide | | | | | | |
| Nitrogen | N₂ | 0 | 0 | 0.007 | 0 | |
| Total | | | | 1 | 2.818 | |

In the described embodiment, the compositions of the flare waste gas streams, i.e., Stream Nos. 1 and 2, were predetermined through laboratory analysis. Flare waste gas samples from each stream vented to the flare 10 are collected and sent to a laboratory for analysis as is known in the art. While this approach is suitable for site specific flares, such as flares located at an upstream production facility, predetermining waste gas stream compositions is not always practicable. For downstream facilities, like certain refineries for instance, predetermining a composition of a waste gas stream is relatively impracticable. This is due to the often rapid variations in the compositions of the streams due to the utilization of multiple sources exhausting or venting to the flare. While real-time waste gas stream composition analysis using, for example, a gas chromatograph is possible and known in the art, it is not presently reasonable for use in determining methane DRE at this time. At present, real time gas composition analysis typically takes around 10 minutes or more before a result can be obtained which is too much lag time for real time methane DRE measurements. As computing speed increases decreasing, if not essentially eliminating, lag time, however, real-time waste gas stream composition analysis may become possible and can be incorporated into the present invention.

As noted above, the DRE of methane is defined as the fraction of methane in a flare gas that is converted to other compounds. Conceptually, if the number of methane molecules going into the flare flame is known, and the number of unburned methane molecules leaving the flare flame, i.e., vented to atmosphere, is known, then the DRE of methane can be calculated using the following formula:

$$DRE of methane = 1 - \frac{number of methane molecules vented to atmosphere}{number of methane molecules going into the flare inlets} \qquad ( Equation 3 )$$

As noted above, a PFTIR instrument 46 measures an amount of energy radiated from the plume P from which all species in the plume can be identified and quantified, in units of ppm×m (concentration level×path length). In other words, the PFTIR instrument 46, which is a radiometer in the described embodiment, is able to determine a signal strength of different carbon-containing species integrated over an optical path through the hot plume P of the flare 10. Signal strength is defined herein as an amount of a carbon-containing species measured using Passive Fourier transform infrared (PFTIR) which is proportional to a concentration of the carbon-containing species integrated over a depth of the hot plume P in an optical path (designated reference numeral 48) of the PFTIR. The PFTIR optical path 48 is generally illustrated in FIG. 2. Signal strength is measured in parts per million×meters (ppm×m).

Since each methane molecule contains one carbon atom, it is advantageous to calculate methane DRE in terms of methane bound carbon atoms. For instance,

$$Fc ( {CH}_{4} , plume ) = \frac{[ {CH}_{4} ]}{[ CO ] + [ {CO}_{2} ] + [ T HC ] + [ soot ]} \qquad ( Equation 4 )$$

where Fc(CH₄, plume) is the methane carbon fraction in the flare plume P, defined by the number of methane-bound carbon atoms in the hot plume of the flare 10, over the total number of carbon atoms in the hot plume of the flare; [CH₄] is the signal strength of the methane measured by PFTIR, which is proportional to the concentration of methane integrated over the depth of the hot plume in the optical path of the PFTIR instrument 46, measured in ppm×m; [CO] is the signal strength of carbon monoxide measured by PFTIR, which is proportional to the concentration of carbon monoxide integrated over the depth of the hot plume in the optical path of the PFTIR instrument, measured in ppm×m; [CO₂] is the signal strength of carbon dioxide measured by PFTIR, which is proportional to the concentration of carbon dioxide integrated over the hot plume in the optical path of the PFTIR instrument, measured in ppm×m; and [THC] is the signal strength of all hydrocarbons measured by PFTIR, which is proportional to the concentration of all hydrocarbons integrated over the depth of the hot plume in the optical path of the PFTIR instrument, measured in ppm×m and weighted by the number of C atoms in the molecules of each of the hydrocarbons. This weighting (see Equation 6) reflected in column 5 of Tables 1 and 2 under the headings “x×F₁(CₓH_{y})” and “x×F₂ (CₓH_{y})”, respectively, wherein the volume fraction for each species is weighted by the number of carbon atoms in the species. The number of carbon atoms in each species is listed in Tables 1 and 2 under the heading “x”.

Since smoking of flares is prohibited by federal law (see, e.g., 40 C.F.R. § 60.18), applications of the disclosed invention are focused on non-smoking flares. Non-smoking flares produce negligible amounts of soot in the hot plume P, so Equation 4 can be simplified to:

$$Fc ( {CH}_{4} , plume ) = \frac{[ {CH}_{4} ]}{[ CO ] + [ {CO}_{2} ] + [ T HC ]} \qquad ( Equation 5 )$$

In Equation 5, [THC] is defined as:

$$[ T HC ] = [ {CH}_{4} ] + 2 [ {C}_{2} {H}_{6} ] + 3 [ {C}_{3} {H}_{8} ] + 4 [ {C}_{4} {H}_{10} ] + 5 [ {C}_{5} {H}_{12} ] + … \qquad ( Equation 6 )$$

Equation 6 can also be written in another format:

$$[ T HC ] = {\sum }_{x = 1}^{x = ∞} x [ {C}_{x} {H}_{y} ] \qquad ( Equation 7 )$$

where x is the number of carbon atoms in a hydrocarbon molecule, and y is the number of hydrocarbon atoms in the hyrdrocarbon molecule. The Cₓ H_{y} nomenclature is used in Tables 1 and 2.

With this, the methane carbon fraction in the flare gas Fc(CH₄, flare gas) can be calculated and defined by the number of methane-bound carbon atoms over the total number of carbon atoms in the flare gases in the following equation. If there is only one flare waste gas stream, i.e., the flare has a single inlet, the methane carbon fraction in the flare gas is:

$$Fc ( {CH}_{4} , flare gas ) = \frac{F ( {CH}_{4} )}{{\sum }_{x = 1}^{x = ∞} xF ( {C}_{x} {H}_{y} )} \qquad ( Equation 8 )$$

where F(CH₄) is the volume fraction of methane in the single flare gas stream; and F(CₓH_{y}) is the volume fraction of each hydrocarbon CₓH_{y}. In practice, when x is greater than a certain threshold (for example, 5), the volume fraction of the hydrocarbon CₓH_{y} may be very small, and therefore could be neglected or not used. This is reflected in Tables 1 and 2 where, for example, the volume fraction for C₆H₁₄ in Table 1 is 0.004 and in Table 2 is 0.017. The upper bound of x can be infinity in theory but in practice a finite integer including, for example, 4-8, can be set as an upper bound depending on the composition of the flare gas.

If there are two flare waste gas streams, as in the described embodiment, the methane carbon fraction in the flare gas can be calculated from the following equation:

$$Fc ( {CH}_{4} , flare gas ) = \frac{{Q}_{1} {F}_{1} ( {CH}_{4} ) + {Q}_{2} {F}_{2} ( {CH}_{4} )}{{Q}_{1} {\sum }_{x = 1}^{x = ∞} {xF}_{1} ( {C}_{x} {H}_{y} ) + {Q}_{2} {\sum }_{x = 1}^{x = ∞} {xF}_{2} ( {C}_{x} {H}_{y} )} \qquad ( Equation 9 )$$

where F₁(CH₄) is the volume fraction of methane in flare waste gas Stream No. 1 (e.g., an HP waste gas stream); Q1 is the volumetric flow rate of flare waste gas Stream No. 1; F₂(CH₄) is the volume fraction of methane in flare waste gas Stream No. 2 (e.g., an LP waste gas stream); and Q2 is the volumetric flow rate of flare waste gas Stream No. 2. It should be noted that these determinations may also be used in flares having more than two waste gas inlets and risers to receive more than two flare waste gas streams. The volumetric flow rate of each flare gas waste stream (e.g., Stream Nos. 1, 2, and 3) may be measured using a volumetric flow meter as shown in FIG. 1 for the two waste gas stream embodiment. Such meters are known for measuring an amount of gas flowing through a pipe by determining a velocity at which the gas flows. In the described embodiment, thermal mass flow meters are used to measure the volumetric flow rate. As is generally known in the art, however, other meter types may also be used to measure volumetric flow rates including, for example, ultrasonic meters, vortex meters, electromagnetic flow meters, turbine flow meters, and positive displacement flow meters, among others.

If there are n flare waste gas streams, for example, the methane carbon fraction in the flare waste gas can generally be calculated from the following equation:

$$Fc ( {CH}_{4} , flare gas ) = \frac{{\sum }_{i = 1}^{n} {Q}_{i} {F}_{i} ( {CH}_{4} )}{{\sum }_{i = 1}^{n} {Q}_{i} {\sum }_{x = 1}^{x = ∞} {x}_{i} {F}_{i} ( {C}_{x} {H}_{y} )} \qquad ( Equation 10 )$$

where Fᵢ(CH₄) is the volume fraction of methane in flare waste gas Stream No. i; Qᵢ is the volumetric flow rate of flare waste gas Stream No. i; Fᵢ(CₓH_{y}) is the volume fraction of hydrocarbon CₓH_{y} in flare waste gas Stream i; x is the number of carbon atom in each molecule of hydrocarbon CₓH_{y}, n is the total number of flare waste gas streams, i is the index of the flare waste gas streams.

In tandem flares, for example, the total number of flare waste gas streams is two. There is typically an HP waste gas stream and an LP waste gas stream. If the HP waste gas stream is designated flare waste gas Stream No. 1 and the LP waste gas stream is designated as flare waste gas Stream No. 2, then, n is 2 in Equation 10. In another example, a flare could have three inlets and three risers to receive three separate flare waste gas streams including, for example, an HP waste gas stream, an MP (medium-pressure) waste gas stream, and an LP waste gas stream. In this second example, n is 3 in Equation 10.

With the methane carbon fraction in the flare plume P, Fc(CH₄, plume), and the methane carbon fraction in the flare gas, Fc(CH₄, flare gas(es)), determined, methane DRE can be calculated from the following equation:

$$Methane DRE = 1 - \frac{Fc ( {CH}_{4} , plumes )}{Fc ( {CH}_{4} , flare gas )} \qquad ( Equation 11 )$$

While Equation 10 may be used for all types of flares regardless of their total number n of flare waste gas streams, Equation 9 may be used in lieu of Equation 10 to determine a methane carbon fraction in flares having two waste gas streams. In one such example, a composition of an HP waste gas stream is predetermined in a laboratory and its gases are shown in Table 1 and a composition of an LP waste gas stream is predetermined and its gases are shown in Table 2. Specifically, the Volume Fraction F₁ (CₓH_{y}) and Volume Fraction F₂ (CₓH_{y}) represent the composition of the respective waste gas streams as determined by the laboratory in the described method.

In addition, an HP waste gas volumetric flow rate is measured during a PFTIR test period (e.g., a period of 3 minutes of relatively steady flow rate) and is 6,079 million standard cubic feet/day or MSCFD. Similarly, an LP waste gas volumetric flow rate is measured during the PFTIR test period and is 34 MSCFD. Utilizing Equation 9 for this example wherein n=2, as noted above, a methane carbon fraction in the respective flare waste gases can be calculated as follows:

$$Fc ( {CH}_{4} , flare gases ) = \frac{( 6 , 079 \times 0.431 ) + ( 34 \times 0.06 )}{( 6 , 079 \times 1.553 ) + ( 34 \times 2.818 )} = 0.428$$

The values 0.431 and 1.553 are taken from Table 1 and the values 0.06 and 2.818 are taken from Table 2.

In operation, the described invention is capable of determining a methane destruction and removal efficiency or methane DRE of an elevated flare 10 receiving at least one waste gas stream and having a flame F and a plume P. The described flare 10 operates in its customary manner with a high-pressure waste gas stream 12a conducted to the flare through an HP flow meter 42 and a low-pressure waste gas stream 14a conducted to the flare through an LP flow meter 44 as shown in FIG. 1. The HP and LP flow rates are determined by the HP and LP flow meters 42, 44 for a period of time and communicated to a computing device 50 or processing unit/device via wired or wireless technology, as is known in the art. Outputs from the flow meters are used in the step of determining a methane carbon fraction for the at least one waste gas stream utilizing equations 8, 9, or 10 as described above.

It should be noted that the computing device 50 may include, but is not limited to, a personal computer, mobile device such as a mobile phone or smartphone, a workstation, a tablet computer, an embedded system, or any other computing device capable of processing data. Such a computing device may include, but is not limited to, a device having a processor and a computer-readable medium coupled to the computing device (e.g., memory) for executing and storing instructions including, for example, the formulas described herein which, when executed by the computing device, cause the computing device to perform specific operations. Such a computing device may include software, firmware, and/or hardware including, for instance, Microsoft Excel brand software which may be utilized to store data and process the data in accordance with the formulas described above. Of course, other known off the shelf software applications may be used for receiving, storing, and/or processing the data. The computing device may also have multiple processors and multiple shared or separate memory components. Software may include one or more applications and an operating system. Hardware can include, but is not limited to, a processor, memory and a graphical user interface display. An optional input device 52, such as a mouse, a keyboard, or a touch screen, may be used.

In addition to the outputs from the flow meters, a predetermined gas composition for each of the at least one waste gas stream is utilized in the step of determining a methane carbon fraction for the at least one waste gas stream. As described above, the gas composition of each waste gas stream is predetermined through laboratory analysis and may be input utilizing input device 52. However, the predetermined gas composition may be acquired through the step of determining, in substantially real time, the gas composition for each of the at least one waste gas stream and provided to the computing device 50 via wired or wireless technology. Whether predetermined from a lab or through real time analysis, the gas composition for each of the at least one waste gas stream is provided to the computer device 52 utilized in the step of determining a methane carbon fraction for the at least one waste gas stream utilizing equations 8, 9, or 10.

As illustrated in FIG. 2, the described invention further utilizes a PFTIR radiometer 46 during operation to remotely sense a signal strength for each carbon-containing species in the plume P of the flare 10. As is known in the art, the PFTIR radiometer 46 uses passive Fourier transform infrared remote sensing over the period of time to determine a signal strength of each different carbon-containing species in the plume P. Output data including the signal strengths is provided to the computing device 50 via wired or wireless technology for use in the step of determining a methane carbon fraction for the plume P utilizing equations 4 or 5 as described above.

With the calculated methane carbon fraction for each of the at least one waste gas stream and the calculated methane carbon fraction for the plume, the methane DRE may be calculated by the processing utilizing equation 11 as described above. The methane DRE may be stored in the computing device memory, printed to an external printer, and/or otherwise communicated to a remote location for further utilization.

In summary, numerous benefits result from the devices for measuring methane DRE for air assistance ground flares and related methods. The foregoing has been presented for purposes of illustration and description. It is not intended to be exhaustive or to limit the embodiments to the precise form disclosed. Obvious modifications and variations are possible in light of the above teachings. For example, the described tandem flare 10 may operate at times with a single waste gas stream flowing in which case it would not be required to measure and/or utilize measurements relating to volumetric flow rates of the waste gas stream(s). All such modifications and variations are within the scope of the appended claims when interpreted in accordance with the breadth to which they are fairly, legally and equitably entitled.

What is claimed:

1. A method of determining a methane destruction and removal efficiency (DRE) of an elevated flare receiving at least one waste gas stream and having a flame and a plume, comprising the steps of:
determining, by a processing unit, a signal strength for each carbon-containing species in the plume using passive Fourier transform infrared remote sensing over a period of time;
measuring a volumetric flow rate for each of the at least one waste gas stream during the period of time;
calculating, by the processing unit, a methane carbon fraction for the at least one waste gas stream using a predetermined gas composition for each of the at least one waste gas stream;
calculating, by the processing unit, a methane carbon fraction for the plume; and
calculating, by the processing unit, the methane DRE using the calculated methane carbon fraction for each of the at least one waste gas stream and the calculated methane carbon fraction for the plume.

2. The method of determining a methane DRE of claim 1, wherein the at least one waste gas stream is conducted to the flame at least partially through the flare.

3. The method of determining a methane DRE of claim 1, further comprising the step of determining a gas composition for each of the at least one waste gas stream.

4. The method of determining a methane DRE of claim 1, wherein the step of calculating a methane carbon fraction for the at least one waste gas stream, by the processing unit, utilizes Equation 8 if the at least one waste gas stream includes a single waste gas stream, Equation 9 if the at least one waste gas stream includes two waste gas streams, and Equation 10 if the at least one waste gas stream includes more than two waste gas streams.

5. The method of determining a methane DRE of claim 1, wherein the step of calculating a methane carbon fraction for the plume, by the processing unit, utilizes Equation 5.

6. The method of determining a methane DRE of claim 1, wherein the step of determining a signal strength for each carbon-containing species in the plume includes the steps of measuring radiation data based on radiation from the plume and a background radiance, calculating, by the processing unit, a difference of a measured radiance of the plume and a measured background radiance, and calculating, by the processing unit, based on the difference, a transmissivity of the plume such that the calculated transmissivity of the plume is defined for flare combustion temperatures.

7. The method of determining a methane DRE of claim 6, wherein the step of determining a signal strength for each carbon-containing species in the plume includes the steps of calculating a correction factor based on atmospheric transmissivity, and wherein the step of calculating transmissivity of the plume further includes the step of calculating, based on the calculated correction factor, the transmissivity of the plume.

8. A method of determining a methane destruction and removal efficiency (DRE) of an elevated flare receiving at least two waste gas streams and having a flame and a plume, utilizing a computing device, comprising the steps of:
determining a gas composition for each of the at least one waste gas stream;
determining a signal strength for each carbon-containing species in the plume using passive Fourier transform infrared remote sensing;
measuring a volumetric flow rate for each of the at least two waste gas streams;
calculating a methane carbon fraction for the at least one waste gas stream using the determined gas composition for each of the at least two waste gas streams;
calculating a methane carbon fraction for the plume; and
calculating the methane DRE using the calculated methane carbon fraction for each of the at least two waste gas streams and the calculated methane carbon fraction for the plume.

9. The method of determining a methane DRE of claim 8, wherein each of the at least two waste gas streams is conducted to the flame at least partially through the flare.

10. The method of determining a methane DRE of claim 9, further comprising the step of mixing the at least two waste gas streams with air conducted through the flare.

11. The method of determining a methane DRE of claim 8, wherein the step of determining a gas composition for each of the at least two waste gas streams includes the steps of collecting waste gas samples for each of the at least two waste gas streams and analyzing the collected waste gas samples before performing the step of determining a signal strength for each carbon-containing species in the plume using passive Fourier transform infrared remote sensing.

12. The method of determining a methane DRE of claim 8, wherein the step of calculating a methane carbon fraction for the at least two waste gas streams utilizes Equation 9 if the at least two waste gas streams include two waste gas streams and Equation 10 if the at least two waste gas streams include more than two waste gas streams.

13. The method of determining a methane DRE of claim 12, wherein the step of calculating a methane carbon fraction for the plume utilizes Equation 5.

14. The method of determining a methane DRE of claim 8, wherein the step of determining a signal strength for each carbon-containing species in the plume includes the steps of measuring radiation data based on radiation from the plume and a background radiance, and calculating a transmissivity of the plume based on a calculated difference of a measured radiance of the plume and a measured background radiance.

15. A system comprising:
a computing device;
a computer-readable medium coupled to the computing device having instructions stored thereon which, when executed by the computing device, cause the computing device to perform operations comprising:
calculating a methane carbon fraction for the plume using a determined signal strength for each carbon-containing species in the plume using passive Fourier transform infrared remote sensing over a period of time;
calculating a methane carbon fraction for at least one waste gas stream conducted at least partially through an elevated flare having a flame and a plume using a predetermined gas composition for each of the at least one waste gas stream and a measured volumetric flow rate for each of the at least one waste gas stream over the period of time; and
calculating the methane DRE using the calculated methane carbon fraction for each of the at least one waste gas stream and the calculated methane carbon fraction for the plume.

16. The system of claim 15, further comprising a flow meter for each of the at least one waste gas stream for measuring a volumetric flow rate for each of the at least one waste gas stream.

17. The system of claim 16, further comprising an PFTIR instrument for determining a signal strength for each carbon-containing species in the plume using passive Fourier transform infrared remote sensing over a period of time.

18. The system of claim 15, wherein the operation of calculating a methane carbon fraction for the at least one waste gas stream utilizes Equation 8 if the at least one waste gas stream includes a single waste gas stream, Equation 9 if the at least one waste gas stream includes two waste gas streams, and Equation 10 if the at least one waste gas stream includes more than two waste gas streams.

19. The system of claim 15, wherein the operation of calculating a methane carbon fraction for the plume, by the processing unit, utilizes Equation 5.
